(19) United States
(12) Patent Application Publication
(10) Pub. No.: US 2025/0318040 A1
(43) Pub. Date: Oct. 9, 2025

(54) PARTICLE BEAM APPARATUS, CALCULATION DEVICE FOR TRANSPORT ROUTE ADJUSTMENT, AND METHOD FOR MANUFACTURING PARTICLE BEAM APPARATUS

(71) Applicant: SUMITOMO HEAVY INDUSTRIES, LTD., Tokyo (JP)

(72) Inventor: Shu NAKAJIMA, Niihama-shi (JP)

(21) Appl. No.: 19/246,486

(22) Filed: Jun. 23, 2025

Related U.S. Application Data
(63) Continuation of application No. PCT/JP2024/001133, filed on Jan. 17, 2024.

(30) Foreign Application Priority Data
Jan. 19, 2023 (JP) 2023-006757

(51) Int. Cl. H05H 7/00

(52) U.S. Cl. CPC H05H 7/001; H05H 2007/002; H05H 2007/005; H05H 2007/008

(57) ABSTRACT

A particle beam apparatus includes: a transport route that guides a particle beam for irradiating an irradiation target to an irradiator; a beam adjustment unit that adjusts a beam size and a beam position of the particle beam; a calculation unit; a storage unit; and an optimization processing unit that searches for a parameter candidate value of the beam adjustment unit, in which the calculation unit adjusts the beam adjustment unit by using the parameter candidate value searched by the optimization processing unit.

### CROSS-REFERENCE TO RELATED APPLICATIONS

This is a bypass continuation of International PCT Application No. PCT/JP2024/001133, filed on Jan. 17, 2024, which claims priority to Japanese Patent Application No. 2023-006757, filed on Jan. 19, 2023, which are incorporated by reference herein in their entirety.

### BACKGROUND

### Technical Field

Certain embodiments of the present invention relate to a particle beam apparatus, a calculation device for transport route adjustment, and a method for manufacturing a particle beam apparatus.

### Description of Related Art

As a particle beam apparatus that performs treatment by irradiating an affected part of a patient with a particle beam, for example, an apparatus described in the related art is known. The particle beam apparatus described in the related art includes a transport route for adjusting a beam size and a beam position of the particle beam in a beam adjustment unit and for transporting the particle beam.

### SUMMARY

According to an embodiment of the present invention, there is provided a particle beam apparatus including: a transport route that guides a particle beam for irradiating an irradiation target to an irradiator; a beam adjustment unit that adjusts a beam size and a beam position of the particle beam; a calculation unit; a storage unit; and an optimization processing unit that searches for a parameter candidate value of the beam adjustment unit, in which the calculation unit adjusts the beam adjustment unit by using the parameter candidate value searched by the optimization processing unit.

According to another embodiment of the present invention, there is provided a calculation device for transport route adjustment, which is used for manufacturing a particle beam apparatus that includes a transport route for guiding a particle beam for irradiating an irradiation target to an irradiator and a beam adjustment unit that adjusts a beam size and a beam position of the particle beam, the calculation device including: a calculation unit; a storage unit; and an optimization processing unit that searches for a parameter candidate value of the beam adjustment unit, in which the calculation unit adjusts the beam adjustment unit by using the parameter candidate value searched by the optimization processing unit.

According to still another embodiment of the present invention, there is provided a method for manufacturing a particle beam apparatus that includes a transport route for guiding a particle beam for irradiating an irradiation target to an irradiator and a beam adjustment unit that adjusts a beam size and a beam position of the particle beam, the method including: searching for a parameter candidate value of the beam adjustment unit by using an optimization unit that adjusts the beam size and the beam position; and adjusting the beam adjustment unit by using the searched parameter candidate value.

### BRIEF DESCRIPTION OF THE DRAWINGS

FIG. 1 is a schematic configuration diagram showing a particle beam treatment apparatus according to an embodiment of the present disclosure.

FIGS. 2A and 2B are diagrams showing an example of a transport route.

FIG. 3 is a flowchart showing an example of processing of a calculation device for transport route adjustment.

FIG. 4 is a flowchart showing an example of the processing of the calculation device for transport route adjustment.

FIG. 5 is a block diagram of the calculation device for transport route adjustment.

FIG. 6 is a diagram for explaining an example of optimization means.

FIG. 7 is a diagram for explaining an example of the optimization means.

FIGS. 8A and 8B are diagrams for explaining an example of the optimization means.

FIGS. 9A and 9B are diagrams showing a simulation result by Bayesian optimization.

FIGS. 10A and 10B are diagrams showing the simulation result by the Bayesian optimization.

FIG. 11 is a diagram showing the simulation result by the Bayesian optimization.

FIGS. 12A and 12B are diagrams showing a simulation result by a Nelder-Mead method.

FIGS. 13A and 13B are diagrams showing the simulation result by the Nelder-Mead method.

FIG. 14 is a diagram showing the simulation result by the Nelder-Mead method.

FIG. 15 is a flowchart showing an example of processing of a calculation device for transport route adjustment according to a modification example.

### DETAILED DESCRIPTION

When manufacturing the particle beam apparatus, it is necessary to adjust the beam adjustment unit of the transport route to obtain a desired beam size and beam position. However, since the beam adjustment unit includes a plurality of electromagnets or the like, it is necessary to try irradiation, measurement, and parameter adjustment multiple times. In the related art, there is a problem in that it takes time to adjust the transport route due to an increase in the number of trial times. In addition, when the trial of the combination of multiple times is performed, there is a possibility that a duct of the transport route is significantly activated, the duct is melted or destroyed, and a leaked dose is increased.

Therefore, it is desirable to provide a particle beam apparatus, a calculation device for transport route adjustment, and a method for manufacturing a particle beam apparatus, in which it is possible to perform transport route adjustment in a short time with a small number of trial times.

In the particle beam apparatus, when the optimization processing unit searches for the parameter candidate value of the beam adjustment unit, an optimal parameter for obtaining a desired beam size and beam position can be obtained with a small number of trial times. Therefore, in the particle beam apparatus, when performing the adjustment of the beam size and beam position in the transport route for the particle beam at any time, such as during maintenance, it is possible to perform the adjustment of the transport route in a short time with a small number of trial times.

When the optimization processing unit searches for the parameter candidate value of the beam adjustment unit, an optimal parameter for obtaining a desired beam size and beam position can be obtained with a small number of trial times. Therefore, when manufacturing the particle beam apparatus, by using the calculation device for transport route adjustment, it is possible to perform the adjustment of the transport route in a short time with a small number of trial times.

In the method for manufacturing a particle beam apparatus, the parameter candidate value of the beam adjustment unit is searched by the optimization unit that adjusts the beam size and the beam position. In this manner, when the parameter candidate value of the beam adjustment unit is searched by the optimization means, an optimal parameter for obtaining a desired beam size and beam position can be obtained with a small number of trial times. In this manner, by adjusting the beam adjustment unit by using the searched parameter candidate value, it is possible to perform the adjustment of the transport route in a short time with a small number of trial times. In addition, the manufacturing time of the particle beam apparatus can be shortened, and a burden on the apparatus and a work burden can be reduced.

Hereinafter, a particle beam treatment apparatus according to an embodiment of the present disclosure will be described with reference to the accompanying drawings. In the description of the drawings, the same elements will be denoted by the same reference numerals, and overlapping description will be omitted.

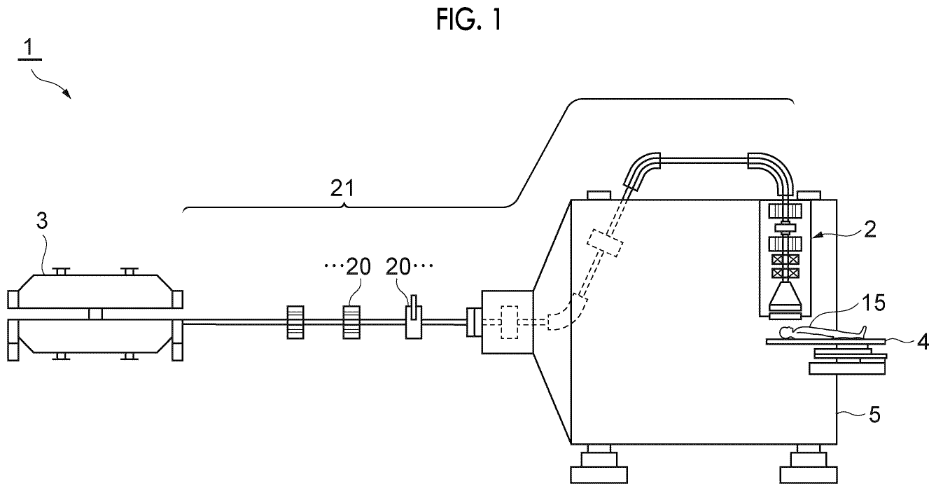

FIG. 1 is a schematic configuration diagram showing a particle beam treatment apparatus 1 according to an embodiment of the present disclosure. The particle beam treatment apparatus 1 is a system that is used for cancer treatment or the like by radiation therapy. The particle beam treatment apparatus 1 includes an accelerator 3 that accelerates charged particles generated by an ion source unit and emits the charged particles as a particle beam, an irradiator 2 that irradiates an irradiation target with the particle beam, and a transport route 21 for transporting the particle beam emitted from the accelerator 3 to the irradiator 2. The irradiator 2 is mounted on a rotating gantry 5 provided to surround a treatment table 4. The irradiator 2 is made to be rotatable around the treatment table 4 by the rotating gantry 5. However, the particle beam treatment apparatus 1 is not limited to the system as shown in FIG. 1, and the present disclosure is applicable to any particle beam treatment apparatus having a transport route for transporting a particle beam B, and can also be applied to a BNCT apparatus.

The accelerator 3 is a device that accelerates charged particles to emit the particle beam B having intensity set in advance. As the accelerator 3, for example, a cyclotron, a synchrocyclotron, or the like can be given. The particle beam B generated by the accelerator 3 is transported to the irradiator 2 by the transport route 21.

The irradiator 2 is for irradiating a tumor (an irradiation target) 14 inside the body of a patient 15 with the particle beam B. The particle beam B is obtained by accelerating charged particles at a high speed, and as the particle beam B, for example, a proton beam, a heavy particle (heavy ion) beam, a particle beam, or the like can be given. Specifically, the irradiator 2 is a device that irradiates the tumor 14 with the particle beam B emitted from the accelerator 3 that accelerates charged particles generated by an ion source (not illustrated) and transported by the transport route 21. The irradiator 2 includes a scanning electromagnet and various types of monitors. In addition, a location where the particle beam B is transported in the irradiator 2 is also included in the transport route 21, and an electromagnet or the like provided in the irradiator 2 is also included in a beam adjustment unit 20. The irradiator 2 performs the irradiation of the particle beam B by a scanning method. However, the irradiation method of the irradiator 2 is not limited to the scanning method.

The transport route 21 connects the accelerator 3 and an exit port of the irradiator 2 to transport the particle beam emitted from the accelerator 3 to the irradiator 2. The transport route 21 includes the beam adjustment unit 20 that adjusts a beam size, a beam position, beam symmetry, and a transmission efficiency of the particle beam B. The beam adjustment unit 20 includes a plurality of electromagnets. The beam adjustment unit 20 includes a quadrupole electromagnet that adjusts a beam size, a bending electromagnet that adjusts a beam position, and the like. The beam adjustment unit 20 adjusts the beam position such that the particle beam B passes through the center inside a duct. The beam adjustment unit 20 adjusts the beam size such that the particle beam B does not collide with the duct due to the spread of the particle beam B.

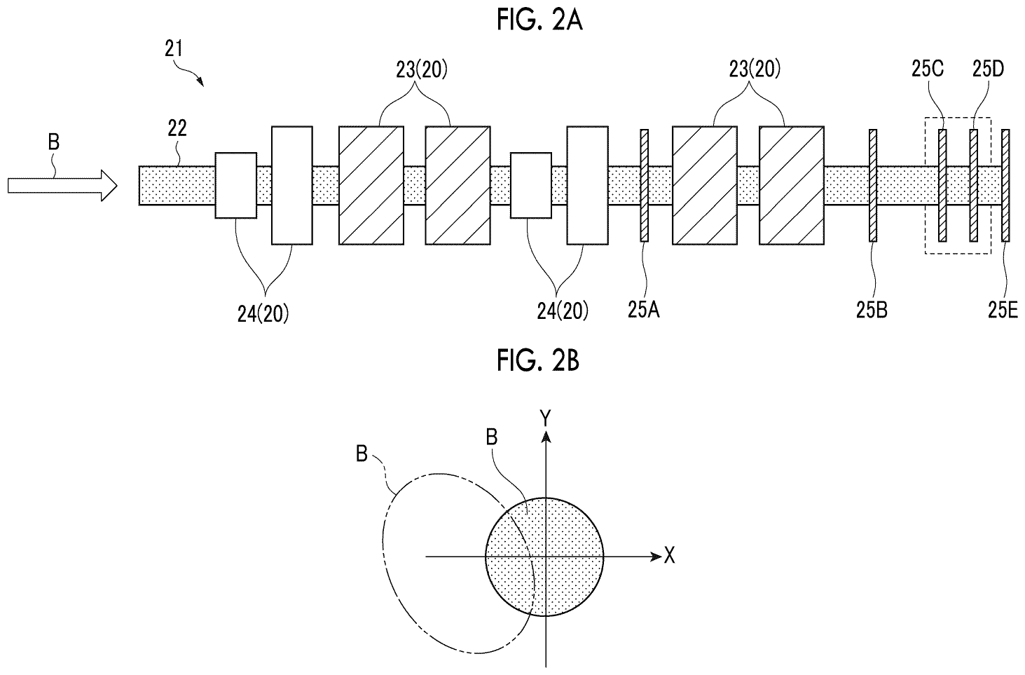

In the following description, there is a case where the transport route 21 is described in a simplified manner as shown in FIG. 2A. The transport route 21 shown in FIG. 2A includes a duct 22, and a plurality of beam size adjustment electromagnets 23 and a plurality of beam position adjustment electromagnets 24 serving as the beam adjustment unit 20. In addition, the transport route 21 includes monitors 25A, 25B, 25C, 25D, and 25E that confirm the beam size and the beam position. The monitors 25A, 25B, 25C, 25D, and 25E are provided in this order from an upstream side with respect to a flow of the particle beam B. The beam size adjustment electromagnet 23 on a most downstream side is provided on a downstream side of the monitor 25A, and the monitors 25B, 25C, 25D, and 25E are provided on a downstream side of the beam size adjustment electromagnet 23 on the most downstream side. The most downstream monitor 25E is the most important monitor. The monitors 25C and 25D surrounded by a broken line are used to perform feedback control of the particle beam B, and are the next important monitors after the monitor 25E.

FIG. 2B shows a cross section of the particle beam B. As shown in FIG. 2B, an X-axis and a Y-axis are set with respect to the particle beam B. The beam size adjustment electromagnet 23 is adjusted to provide a small, round, and symmetrical beam size. For example, the shape of a large and asymmetric particle beam B as shown by an imaginary line is adjusted to a small regular circle. The beam position adjustment electromagnet 24 performs adjustment such that the center of the particle beam B is aligned with the center of the duct 22 (the intersection point of the X-axis and the Y-axis). For example, the particle beam B deviated from the center as shown by an imaginary line is adjusted to move to the center.

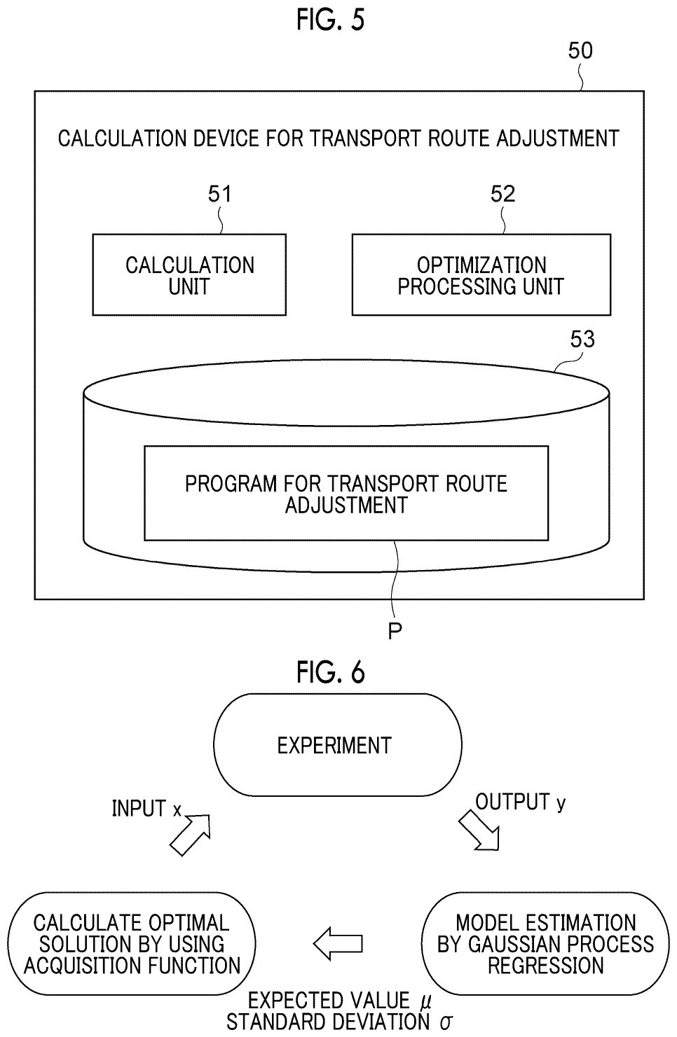

Next, a calculation device for transport route adjustment 50 shown in FIG. 5 will be described. The calculation device for transport route adjustment 50 is a device that adjusts the beam adjustment unit 20 such that each of the beam size and the beam position of the particle beam B is in a desired state. The calculation device for transport route adjustment 50 calculates the parameter of the beam adjustment unit 20 for making each of the beam size and the beam position a desired state. As the parameter of the beam adjustment unit 20, a current value for each of the electromagnets 23 and 24, a current value of a bending electromagnet, or the like can be given. In the present embodiment, the calculation device for transport route adjustment 50 searches for a parameter candidate value of the beam adjustment unit 20 by an optimization unit that adjusts the beam size and the beam position by using artificial intelligence. The calculation device for transport route adjustment 50 is connected to the beam adjustment unit 20 and the monitors 25A, 25B, 25C, 25D, and 25E.

The calculation device for transport route adjustment 50 is configured with hardware including a CPU that performs calculation, a ROM in which various programs are stored, a RAM in which data generated by processing of the CPU is stored, and the like. The calculation device for transport route adjustment 50 includes a calculation unit 51, an optimization processing unit 52, and a storage unit 53. The calculation unit 51 performs various calculations relating to the particle beam B in the transport route 21. The calculation unit 51 is configured by a calculation device that does not have artificial intelligence (AI). The optimization processing unit 52 has artificial intelligence. The storage unit 53 stores various information. The storage unit 53 stores a program for transport route adjustment P. The program for transport route adjustment P is a program for causing the calculation unit 51 and the optimization processing unit 52 to execute a calculation method for transport route adjustment.

The calculation unit 51 performs actual measurement when the irradiation of the particle beam B is performed with respect to an arbitrary current value, and determines whether or not the actual measurement result is a desired design value. The calculation unit 51 sets the current value for each of the electromagnets 23 and 24 of the beam adjustment unit 20, based on the optimization processing of the optimization processing unit 52. The calculation unit 51 calculates the trajectory of the particle beam B, based on the set current value. The calculation unit 51 calculates a difference between the beam size and beam position in each of the monitors 25A, 25B, 25C, 25D, and 25E and a desired design value set in advance, and determines whether or not the difference is within an allowable range. The difference when determining whether or not the difference is within the allowable range is a difference between a simple design value and an actual measurement value.

The optimization processing unit 52 searches for the parameter candidate value of the beam adjustment unit 20 by the optimization unit that adjusts the beam size and the beam position by using artificial intelligence. As shown in FIG. 6, the optimization means of the optimization processing unit 52 estimates a function by Gaussian process regression. The optimization means includes searching for a candidate value by Bayesian optimization.

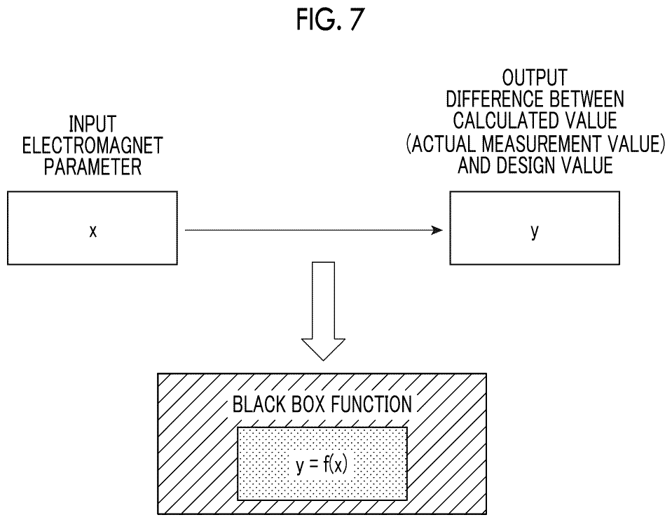

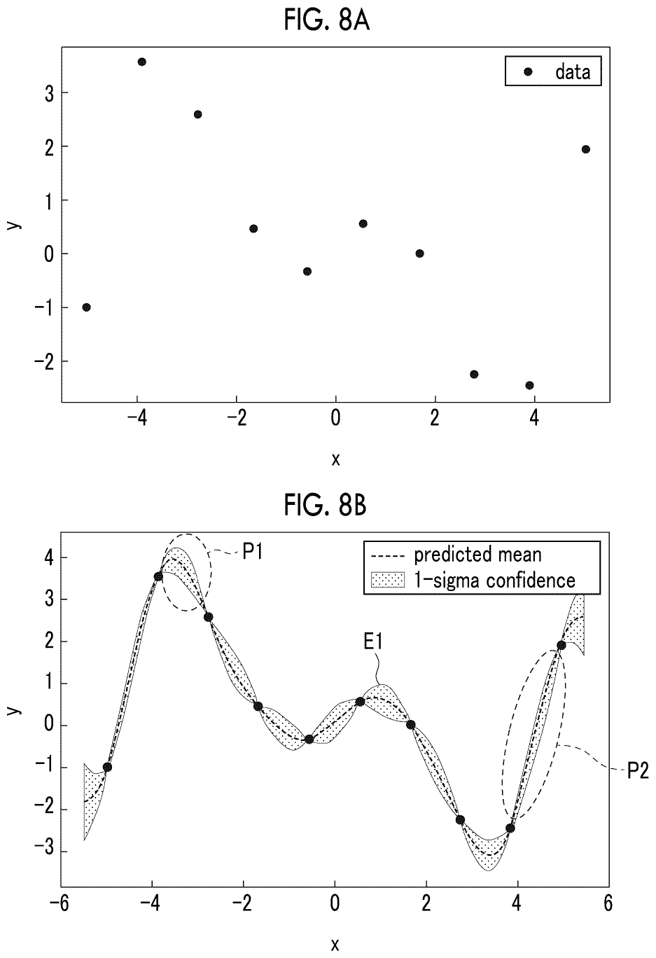

FIG. 6 is a diagram showing a processing image of the Bayesian optimization. As shown in FIG. 6, in the Bayesian optimization, an experiment is performed by an input x, and model estimation by the Gaussian process regression is performed based on an output y. The optimal solution of the input x is calculated by using an acquisition function, and based on an estimation result. The processing is repeated, whereby the input x is optimized. As shown in FIG. 7, a function f(x) which is a black box function is estimated based on a Gaussian process method from the input/output data (x1, y1), . . . , (xN, yN), and the optimal solution is efficiently obtained by maximizing the acquisition function, based on the model. Here, the input x is a parameter of each of the electromagnets 23 and 24, and the output y is a difference between a calculated value (actual measurement value) and a design value. In the estimation result of f(x) by the Gaussian process method, it is obtained in the form of an expected value μ(x) and a standard deviation σ(x). A predicted value of the model may be obtained in the form of probability μ(x)±(x) instead of a value. The optimal solution is where the expected value μ(x) is the maximum. However, there is also a case where the optimal solution is present in a place where the standard deviation σ(x) is large. A value of the next input x is obtained using the acquisition function. FIGS. 8A and 8B show an image of estimating a function by a Gaussian process. For example, in a case where a plurality of input/output data (x, y) as shown in FIG. 8A are obtained, a function f(x) as shown in a graph of a broken line in FIG. 8B is estimated. A region indicated by E1 shows the range of “μ(x)±σ(x)” with respect to the function f(x). A portion indicated by P1 is a portion where the range of the region E1 is wide and the reliability is low. A portion indicated by P2 is a portion where the range of the region E1 is narrow and the reliability is high. In addition, the difference when calculating the acquisition function in the present paragraph is the sum of the weighted squares of the difference between the design value and the actual measurement value (root mean square error).

The acquisition function is a function (α(x)) defined as an appropriate combination of the expected value μ and the standard deviation σ, and is defined, for example, as “Lower Confidence Bound (LCB): α(x)=μ(x)−βσ(x)” or the like. β is a coefficient (exploration weight) applied to σ, and determines a ratio of the exploration to the exploitation. Since the acquisition function reflects the past actual examples, the optimization processing unit 52 can perform the processing using the optimization means as described above by performing learning by artificial intelligence.

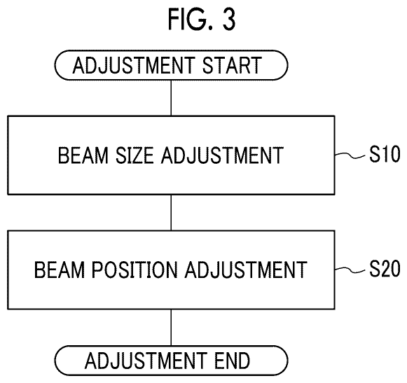

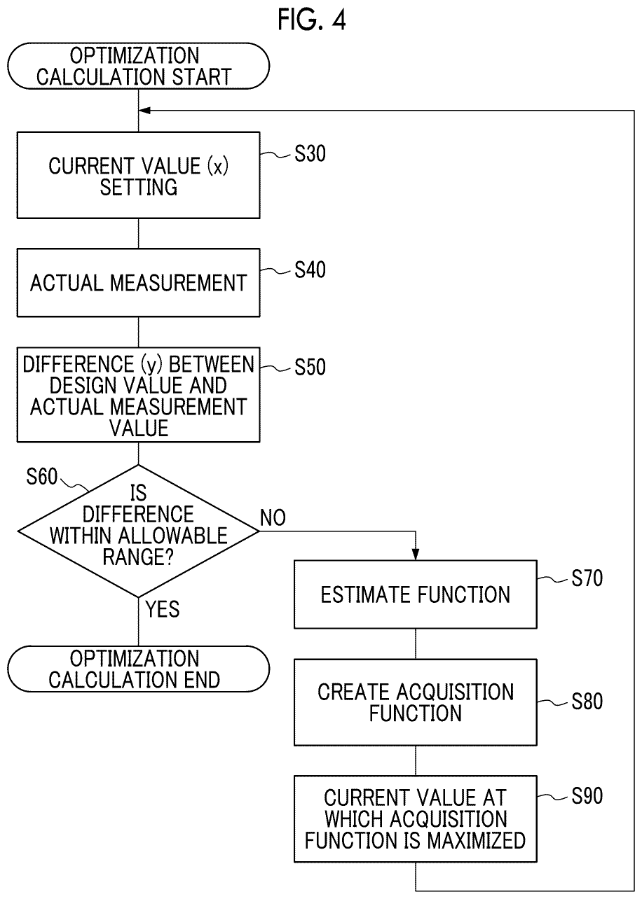

Next, a method for manufacturing the particle beam treatment apparatus 1 according to the present embodiment will be described with reference to FIGS. 3 and 4. FIGS. 3 and 4 show a method when the manufacture of the apparatus configuration of the particle beam treatment apparatus 1 is completed and the adjustment of the beam adjustment unit 20 is performed. FIGS. 3 and 4 are executed by the calculation device for transport route adjustment 50. As shown in FIG. 3, the calculation device for transport route adjustment 50 performs beam size adjustment (step S10). When the beam size adjustment is completed, the calculation device for transport route adjustment 50 performs beam position adjustment (step S20). When the beam position adjustment is completed, the processing shown in FIG. 3 is ended. When the beam size and the beam position are adjusted at the same time, the electromagnets 23 and 24 perform operations having different roles. Therefore, the beam size adjustment and the beam position adjustment are separately performed. Therefore, the beam position adjustment may be performed first, and the beam size adjustment may be performed later.

FIG. 4 is a flowchart showing specific processing of the optimization calculation that is executed in each of the beam size adjustment S10 and the beam position adjustment S20. As shown in FIG. 4, the calculation unit 51 sets the current value for each of the electromagnets 23 and 24 (step S30). Next, the calculation unit 51 acquires information such as the beam size and the beam position, based on the result of actual measurement from the monitor, when the irradiation of the particle beam B is performed based on the set current value (step S40). Next, the calculation unit 51 calculates a difference between the result of actual measurement (actual measurement value) acquired in step S40 and a design value set in advance (step S50). In step S50, a weight may be multiplied after the calculation of the difference at each monitor. The calculation unit 51 determines whether or not the difference calculated in step S50 is within an allowable range (step S60). The difference as referred to herein is a difference between a simple design value and an actual measurement value.

When it is determined in step S60 that the difference is out of the allowable range, the optimization processing unit 52 estimates the function f(x) by the Gaussian process regression (step S70). Next, the optimization processing unit 52 creates the acquisition function α(x) (step S80). Next, the optimization processing unit 52 obtains a current value at which the acquisition function α(x) is maximized as a parameter candidate value of the beam adjustment unit 20 (step S90). When step S90 is completed, the calculation unit 51 repeats the processing from step S30 again, based on the obtained current value.

When it is determined in step S60 that the difference is within the allowable range, the processing shown in FIG. 4 is ended. In the case of the beam size adjustment processing in step S10, the calculation unit 51 adopts a candidate value that satisfies the allowable range for the beam size as the current value of the beam size adjustment electromagnet 23. In the case of the beam position adjustment processing in step S20, the calculation unit 51 adopts a candidate value that satisfies the allowable range for the beam position as the current value of the beam position adjustment electromagnet 24. The calculation device for transport route adjustment 50 may perform the adjustment of the symmetry of the particle beam B and the transmission efficiency by the optimization means by performing the processing shown in FIG. 4.

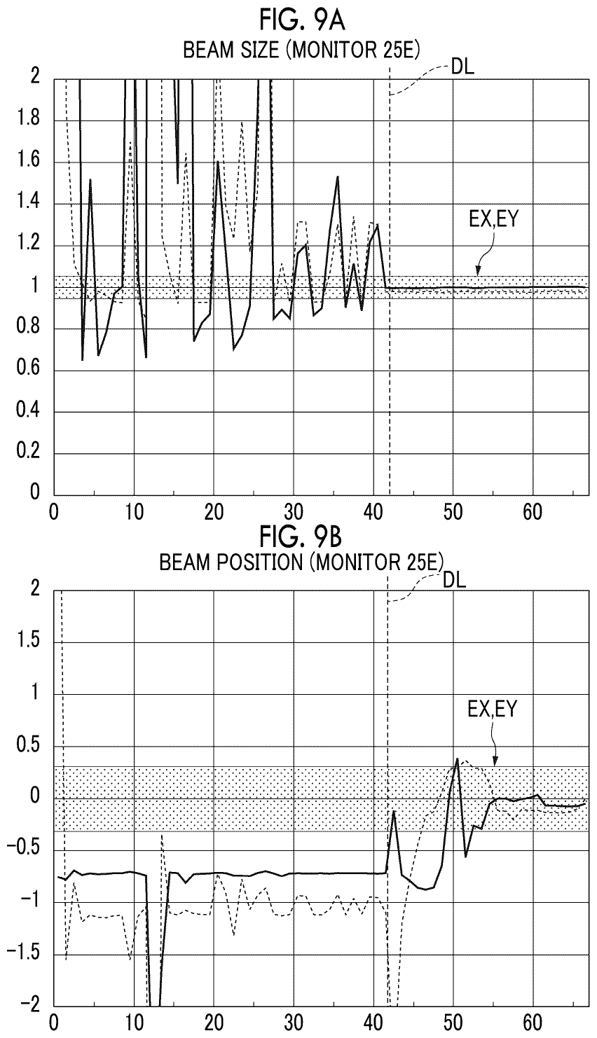

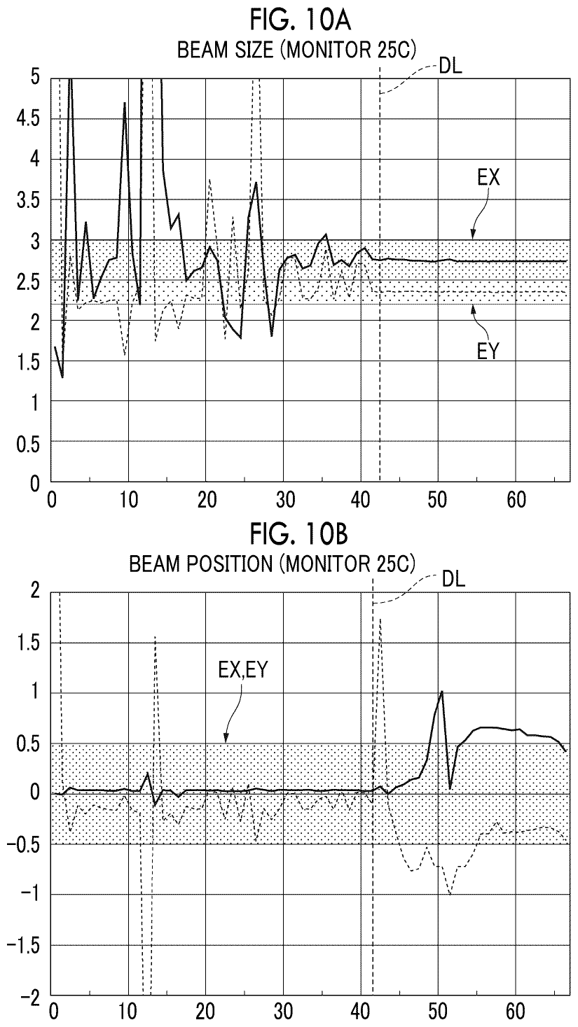

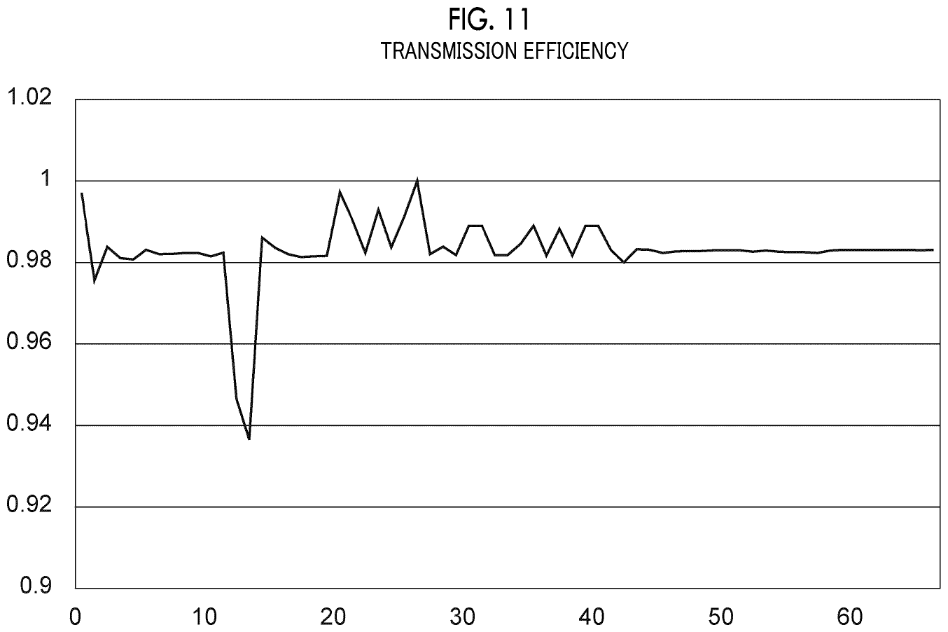

An example of a specific simulation result will be described with reference to FIGS. 9A to 11. In the simulation, trajectory calculation is performed instead of the actual measurement in step S40. The result of the trajectory calculation includes information such as the beam size, the beam position, the symmetry of the beam, and the transmission efficiency. FIG. 9A is a graph showing a transition of the beam size based on the detection result by the monitor 25E. FIG. 9B is a graph showing a transition of the beam position based on the detection result by the monitor 25E. FIG. 10A is a graph showing a transition of the beam size based on the detection result by the monitor 25C. FIG. 10B is a graph showing a transition of the beam position based on the detection result by the monitor 25C. FIG. 11 is a graph showing a transition of the transmission efficiency. The horizontal axis of FIGS. 9A to 11 represents the number of times of searching for a candidate value. The vertical axis of FIGS. 9A and 10A represents the beam size (mm). The vertical axis of FIGS. 9B and 10B represents the beam position (mm). The vertical axis of FIG. 11 represents the transmission efficiency (%). In FIGS. 9A and 9B, and 10A and 10B, a solid line graph indicates the beam size or the beam position in the X-axis direction, and a broken line graph indicates the beam size or the beam position in the Y-axis direction. In addition, FIGS. 9A and 9B, and 10A and 10B show an allowable range EX for the beam size or the beam position in the X-axis direction, and an allowable range EY for the beam size or the beam position in the Y-axis direction. The allowable ranges EX and EY are represented by “design value±allowable value”. When the beam size or the beam position can be adjusted to fall within the allowable ranges EX and EY, the adjustment can be regarded as being completed. The symmetry of the particle beam B can be evaluated based on the smallness of the deviation between the beam size or the beam position in the X-axis direction and the beam size or the beam position in the Y-axis direction.

As shown in FIGS. 9A and 10A, in any of the monitors 25C and 25E, the beam size randomly changes until the number of times of searching reaches approximately 40 times. However, the beam sizes in both the X-axis direction and the Y-axis direction fall within the allowable range EX and EY in a range exceeding approximately 40 times. Therefore, the adjustment of the beam size is completed in approximately 40 times. Therefore, the beam size adjustment may be performed in a region on a negative side with respect to a boundary line DL near the number of times of searching of 40, and the beam position adjustment may be performed in a region on a positive side with respect to the boundary line DL. In this way, as shown in FIGS. 9B and 10B, in any of the monitors 25C and 25E, the beam positions in both the X-axis direction and the Y-axis direction fall within the allowable range EX and EY in the region on the positive side with respect to the boundary line DL by repeating the searching. As shown in FIG. 11, the transmission efficiency also falls within the allowable range after about 40 times.

The operation and effect of the present embodiment will be described.

In the particle beam treatment apparatus 1, when the parameter candidate value of the beam adjustment unit 20 is searched by the optimization processing unit 52, an optimal parameter for obtaining a desired beam size and beam position can be obtained with a small number of trial times. Therefore, in the particle beam treatment apparatus 1, when the adjustment of the beam size and beam position in the transport route for the particle beam is performed at any time, such as during maintenance, the adjustment of the transport route can be performed in a short time with a small number of trial times. For example, in an apparatus having a high charged particle beam dose, such as a boron neutron capture therapy (BNCT) apparatus, it is possible to effectively suppress the amount of the particle beam that is irradiated to the duct during trial and error, and it is possible to suppress the duct from being highly activated, the duct from being melted or destroyed, and a leaked dose from increasing.

The optimization processing unit 52 may estimate a function by the Gaussian process regression. In this case, it is possible to search for the optimal solution in a short time.

The optimization processing unit 52 may include searching for a candidate value by Bayesian optimization. In this case, it is possible to search for the optimal solution in a short time.

The optimization processing unit 52 may adjust the symmetry of the particle beam and the transmission efficiency. In this case, it is possible to obtain a symmetrical particle beam. In addition, by adjusting the transmission efficiency, it is possible to identify the behavior of the particle beam in a place where the monitor is not present.

The calculation device for transport route adjustment 50 is the calculation device for transport route adjustment 50, which is used for manufacturing the particle beam treatment apparatus 1 including the transport route 21 for guiding the particle beam that is irradiated to the irradiation target to the irradiator 2 and the beam adjustment unit 20 that adjusts the beam size and the beam position of the particle beam, the calculation device for transport route adjustment 50 including: the calculation unit 51, the storage unit 53, and the optimization processing unit 52 that searches for a parameter candidate value of the beam adjustment unit 20, in which the calculation unit 51 adjusts the beam adjustment unit 20 by using the parameter candidate value searched by the optimization processing unit 52.

When the optimization processing unit 52 searches for the parameter candidate value of the beam adjustment unit 20, an optimal parameter for obtaining a desired beam size and beam position can be obtained with a small number of trial times. Therefore, when manufacturing the particle beam treatment apparatus 1, by using the calculation device for transport route adjustment 50, it is possible to perform the adjustment of the transport route in a short time with a small number of trial times.

The method for manufacturing the particle beam treatment apparatus 1 is the method for manufacturing the particle beam treatment apparatus 1 including the transport route 21 for guiding the particle beam that is irradiated to the irradiation target to the irradiator 2 and the beam adjustment unit 20 that adjusts the beam size and the beam position of the particle beam, the method includes: searching for a parameter candidate value of the beam adjustment unit by using the optimization unit that adjusts the beam size and the beam position; and adjusting the beam adjustment unit by using the searched parameter candidate value.

In the method for manufacturing the particle beam treatment apparatus 1, the parameter candidate value of the beam adjustment unit 20 is searched by the optimization unit that adjusts the beam size and the beam position. In this manner, when the parameter candidate value of the beam adjustment unit 20 is searched by the optimization means, an optimal parameter for obtaining a desired beam size and beam position can be obtained with a small number of trial times. In this manner, by adjusting the beam adjustment unit 20 by using the searched parameter candidate value, it is possible to perform the adjustment of the transport route 21 in a short time with a small number of trial times. In addition, the manufacturing time of the particle beam treatment apparatus 1 can be shortened, and a burden on the apparatus and a work burden can be reduced.

The calculation method for transport route adjustment is a calculation method for transport route adjustment, which is used for manufacturing the particle beam treatment apparatus 1 that includes the transport route 21 adjusting the beam size and the beam position of the particle beam with the beam adjustment unit 20 and transporting the particle beam, and that irradiates an irradiation target with the particle beam B, the calculation method including: searching for a parameter candidate value of the beam adjustment unit 20 by using the optimization unit that adjusts the beam size and the beam position.

When the parameter candidate value of the beam adjustment unit 20 is searched by the optimization means, an optimal parameter for obtaining a desired beam size and beam position can be obtained with a small number of trial times. Therefore, when manufacturing the particle beam treatment apparatus 1, by using the calculation method for transport route adjustment, it is possible to perform the adjustment of the transport route in a short time with a small number of trial times.

The program for transport route adjustment P is a program for transport route adjustment that is used for manufacturing a particle beam treatment apparatus including a transport route adjusting a beam size and a beam position of the particle beam with the beam adjustment unit 20 and transporting the particle beam, and that irradiates an irradiation target with the particle beam, the program causing a computer to execute the processing of searching for a parameter candidate value of the beam adjustment unit 20 by the optimization unit that adjusts the beam size and the beam position by using artificial intelligence.

When the parameter candidate value of the beam adjustment unit is searched by the optimization means, an optimal parameter for obtaining a desired beam size and beam position can be obtained with a small number of trial times. Therefore, when manufacturing the particle beam treatment apparatus 1, the transport route adjustment can be performed in a short time with a small number of trial times by using the program for transport route adjustment P.

In a case where the adjustment is performed without depending on the embodiment described above, it is necessary to actually measure the particle beam B emitted from the accelerator 3 at the time of the start of the adjustment, and to calculate the initial value of the parameter of the beam adjustment unit 20 to calculate a design value. That is, in facilities to which the particle beam treatment apparatus 1 is applied, the conditions are different for each facility. Therefore, it is necessary to confirm the difference between the actual measurement value, the design value, and the setting value of the initial value for each facility, which requires a lot of time and effort for adjustment. On the other hand, in the present embodiment, the adjustment of the parameter of the beam adjustment unit 20 can be performed such that the beam size and the beam position have the design values, by the learning of the artificial intelligence of the optimization processing unit 52, without measuring the difference between the actual measurement value and the design value for each facility. The optimization processing unit 52 may perform the adjustment of the parameter by reinforcement learning as an optimization method. However, in the case of the reinforcement learning, it is vulnerable to perturbation, and there is a case where adjustment can be performed accurately only within a range of learning. That is, there is a case where it takes time to perform adjustment in a case where the facilities are different from each other, or the accuracy is degraded. On the other hand, by performing the function estimation (Bayesian optimization) by the Gaussian process regression, there is the advantage that it is possible to omit a learning phase such as the reinforcement learning, to efficiently find an optimal solution, and to perform the parameter adjustment by using the same program even when the facilities are different.

The present disclosure is not limited to the embodiment described above.

For example, the optimization means described above performs optimization using only the Bayesian optimization. However, the optimization means may execute a method of searching for a candidate value in a region where an expected value is high, after the searching by the Bayesian optimization. For example, when only the Bayesian optimization is used to search for the candidate value, a value having a large error is also searched. Therefore, there is a case where a region having a large error is also searched regardless of the expected value. Therefore, there is a possibility that the duct may be further activated or damaged due to the beam size becoming too large or the beam position being greatly shifted. The method of searching for the candidate value in the region where the expected value is high is executed in combination, so that the risk described above can be reduced.

The method may use an algorithm in which a derivative is not required. Since the derivative includes a component that is not differentiated, such as the transmission efficiency, it is suitable to use the algorithm in which a derivative is not required.

The method may be a Nelder-Mead method. The method is a local optimization solution method, and depends on an initial value, so that the candidate value can be difficult to fall into a region where an expected value is low.

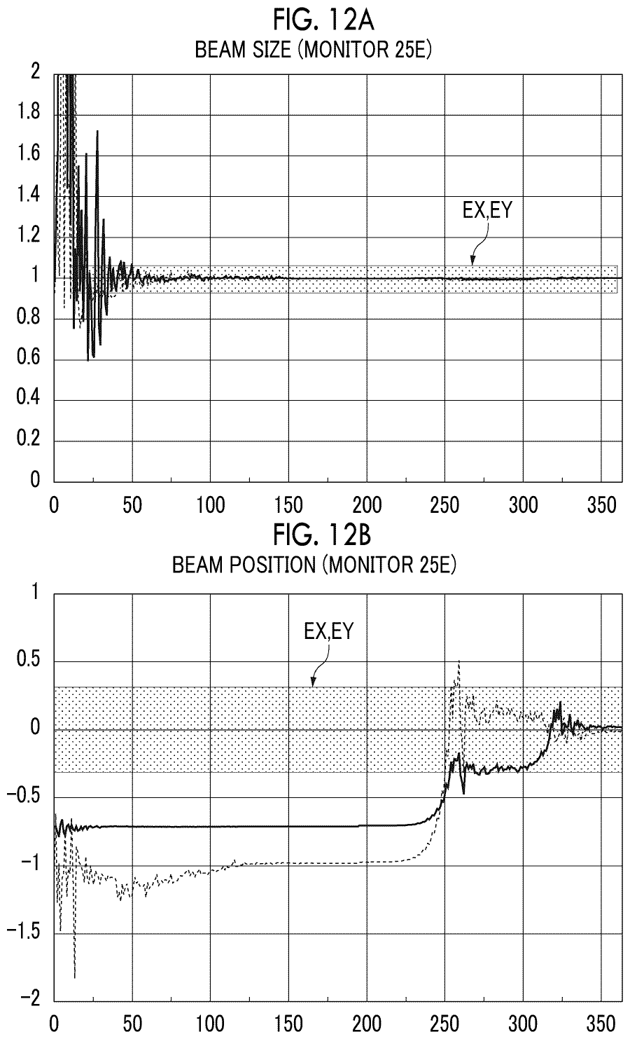

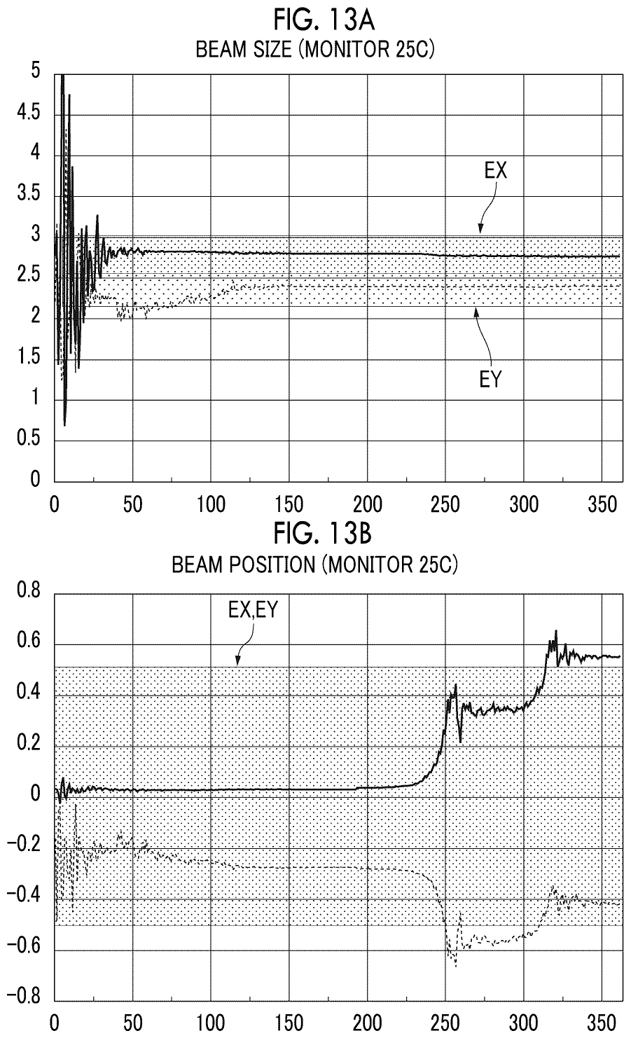

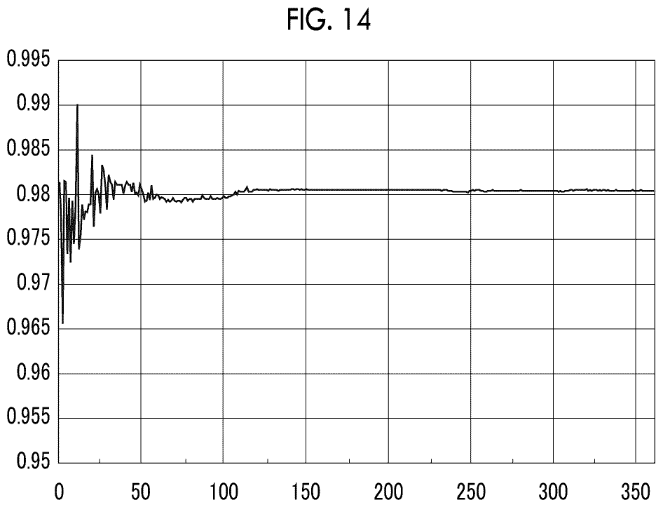

For example, FIGS. 12A to 14 are graphs corresponding to FIGS. 9A to 11, which are created by performing simulation using the Nelder-Mead method. In FIG. 12A, the beam size is within the allowable ranges EX and EY after searching of about 70 times. In addition, in FIG. 12B, searching of 100 or more times is required. However, the number of data in which the value of the beam size or the value of the beam position is out of a region where an expected value is high (a region close to the allowable ranges EX and EY) is small. That is, it is possible to reduce a risk such as the activation of the duct or the damage to equipment.

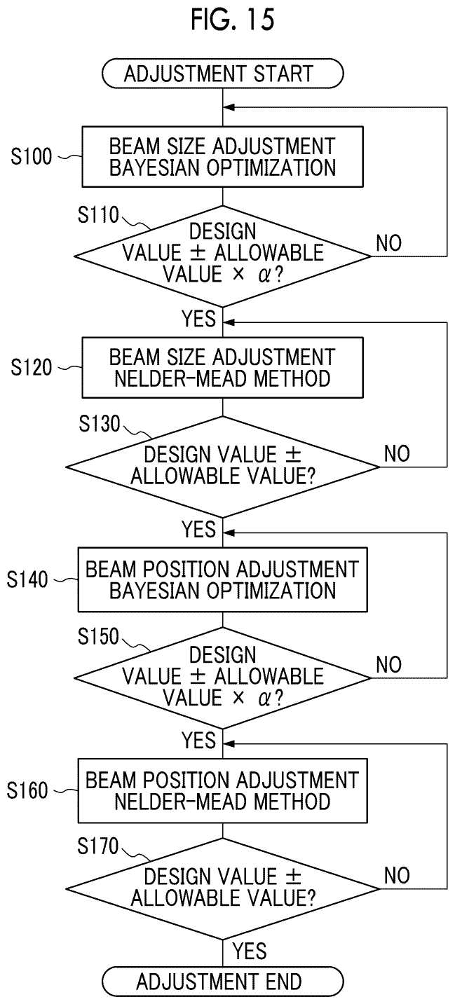

Therefore, as shown in FIG. 15, the adjustment by the Bayesian optimization is performed to quickly bring the candidate value close to the region where an expected value is high, and thereafter, the candidate value may be searched in the region where an expected value is high, by the Nelder-Mead method. First, the calculation device for transport route adjustment 50 performs beam size adjustment by Bayesian optimization (step S100). The calculation device for transport route adjustment 50 determines whether or not the difference between the actual measurement value and the design value is within an allowable range (design value±allowable value×α) (step S110). In a case where the condition is not satisfied, the process returns to S100. In a case where the condition is satisfied, the calculation device for transport route adjustment 50 performs the beam size adjustment by the Nelder-Mead method (step S120). The calculation device for transport route adjustment 50 determines whether or not the difference between the actual measurement value and the design value is within an allowable range (design value±allowable value) (step S130). In a case where the condition is not satisfied, the process returns to S120. In a case where the condition is satisfied, the calculation device for transport route adjustment 50 performs the beam position adjustment by the Bayesian optimization (step S140). The calculation device for transport route adjustment 50 determines whether or not the difference between the actual measurement value and the design value is within an allowable range (design value±allowablevalue×α) (step S150). In a case where the condition is not satisfied, the process returns to S140. In a case where the condition is satisfied, the calculation device for transport route adjustment 50 performs the beam position adjustment by the Nelder-Mead method (step S160). The calculation device for transport route adjustment 50 determines whether or not the difference between the actual measurement value and the design value is within an allowable range (design value±allowable value) (step S170). In a case where the condition is not satisfied, the process returns to S160. In a case where the condition is satisfied, the processing shown in FIG. 15 is ended. In addition, “a” in steps S110 and S150 is a value larger than 1. In this way, in the case of the adjustment by Bayesian optimization, the allowable range is widened, so that the searching in the region where the expected value is high can be quickly performed by the Nelder-Mead method.

The calculation device for transport route adjustment 50 may be included in the particle beam treatment apparatus 1, may be present independently of the particle beam treatment apparatus 1, and may be connected to the beam adjustment unit 20 of the particle beam treatment apparatus 1 in a case where the beam size and the beam position need to be adjusted when the particle beam treatment apparatus 1 is manufactured, and be separated from the particle beam treatment apparatus 1 after the adjustment.

In addition, the calculation device for transport route adjustment 50 described in the embodiment described above is not limited to being applied to the particle beam treatment apparatus 1. The calculation device for transport route adjustment 50 can be applied to a particle beam apparatus having a transport route for transporting the particle beam. For example, in a radio isotope (RI) production apparatus that irradiates a metal or liquid target, which is an irradiation target, with accelerated particles, the adjustment of the beam size and the beam position in the transport route for the particle beam can be performed.

In addition, even after the adjustment at the time of manufacture of the particle beam apparatus, the adjustment of the beam size and the beam position in the transport route for the particle beam may be performed at any time, such as during maintenance.

It should be understood that the invention is not limited to the above-described embodiment, but may be modified into various forms on the basis of the spirit of the invention. Additionally, the modifications are included in the scope of the invention.

What is claimed is:

1. A particle beam apparatus comprising:
a transport route that guides a particle beam for irradiating an irradiation target to an irradiator;
a beam adjustment unit that adjusts a beam size and a beam position of the particle beam;
a calculation unit;
a storage unit; and
an optimization processing unit that searches for a parameter candidate value of the beam adjustment unit,
wherein the calculation unit adjusts the beam adjustment unit by using the parameter candidate value searched by the optimization processing unit.

2. The particle beam apparatus according to claim 1, further comprising:
an accelerator that accelerates charged particles generated by an ion source unit and emits the charged particles as a particle beam.

3. The particle beam apparatus according to claim 1, wherein the irradiator is mounted on a rotating gantry provided to surround a treatment table, and is rotatable around the treatment table by the rotating gantry.

4. The particle beam apparatus according to claim 2, wherein the irradiator irradiates the irradiation target with the particle beam emitted from the accelerator and transported by the transport route.

5. The particle beam apparatus according to claim 1, wherein the beam adjustment unit includes a plurality of electromagnets for adjusting the beam size and the beam position.

6. The particle beam apparatus according to claim 1, wherein the transport route includes a duct, a plurality of beam size adjustment electromagnets, a plurality of beam position adjustment electromagnets, and a plurality of monitors for confirming the beam size and the beam position.

7. The particle beam apparatus according to claim 6, wherein the plurality of monitors are provided in order from an upstream side to a downstream side with respect to a flow of the particle beam, the beam size adjustment electromagnet on a most downstream side is provided on a downstream side of a most upstream monitor, and remaining monitors are provided on a downstream side of the beam size adjustment electromagnet on the most downstream side.

8. The particle beam apparatus according to claim 1, wherein the optimization processing unit estimates a function by Gaussian process regression.

9. The particle beam apparatus according to claim 8, wherein the optimization processing unit includes searching for the candidate value by Bayesian optimization.

10. The particle beam apparatus according to claim 9, wherein the optimization processing unit executes a method of searching for the candidate value in a region where an expected value is high, after the searching by the Bayesian optimization.

11. The particle beam apparatus according to claim 10, wherein the method uses an algorithm in which a derivative is not required.

12. The particle beam apparatus according to claim 11, wherein the method is a Nelder-Mead method.

13. The particle beam apparatus according to claim 1, wherein the optimization processing unit adjusts symmetry of the particle beam and a transmission efficiency.

14. A calculation device for transport route adjustment, which is used for manufacturing a particle beam apparatus that includes a transport route for guiding a particle beam for irradiating an irradiation target to an irradiator and a beam adjustment unit that adjusts a beam size and a beam position of the particle beam, the calculation device comprising:
a calculation unit;
a storage unit; and
an optimization processing unit that searches for a parameter candidate value of the beam adjustment unit,
wherein the calculation unit adjusts the beam adjustment unit by using the parameter candidate value searched by the optimization processing unit.

15. A method for manufacturing a particle beam apparatus that includes a transport route for guiding a particle beam for irradiating an irradiation target to an irradiator and a beam adjustment unit that adjusts a beam size and a beam position of the particle beam, the method comprising:
searching for a parameter candidate value of the beam adjustment unit by using an optimization unit that adjusts the beam size and the beam position; and
adjusting the beam adjustment unit by using the searched parameter candidate value.
